(19) United States
(12) Patent Application Publication
(10) Pub. No.: US 2020/0113954 A1
(43) Pub. Date: Apr. 16, 2020

(54) PHARMACEUTICAL AND FOOD COMPOSITION FOR THE TREATMENT OF VAGINAL AND INTESTINAL DYSBIOSIS

(71) Applicant: UNIDERM FARMACEUTICI S.R.L., Rome (IT)

(72) Inventors: Giorgio CHIOZZA, Rome (IT); Francesco DE SETA, Rome (IT); Sara OLMO, Rome (IT); Anna SABATO, Rome (IT); Moira MIGLIUOLO, Rome (IT)

(21) Appl. No.: 16/654,628

(22) Filed: Oct. 16, 2019

(30) Foreign Application Priority Data
Oct. 16, 2018 (IT) 102018000009490

(51) Int. Cl. A61K 35/747; A61K 31/4045; A61K 35/741; A61K 35/745; A61K 31/7048; A61K 31/4196; A61K 31/4164; A61K 9/00; A61K 36/53; A61P 31/10

(52) U.S. Cl. CPC A61K 35/747; A61K 31/4045; A61K 35/741; A61K 35/745; A61K 31/7048; A61P 31/10; A61K 31/4164; A61K 9/0053; A61K 9/0034; A61K 36/53; A61K 31/4196

(57) ABSTRACT

The invention refers to the use of pharmaceutical and food compositions containing Melatonin, a yeast (belonging to the Saccharomyces family) and a bacterium (belonging to the Lactobacillus, Bifidobacterium, Streptococcus families) for use in the treatment of disorders associated with vaginal and intestinal diseases caused by altered balance in the flora (dysbiosis), which are useful for reducing inflammation due to local and systemic infections caused by the excessive growth of pathogenic microorganisms.

### TECHNICAL FIELD OF THE INVENTION

The present invention relates to pharmaceutical and food compositions for the treatment of vaginal and intestinal dysbiosis. These compositions include, in combination, Lactobacilli, also referred to below as lactic acid bacteria, yeasts and melatonin. The present invention relates in particular to use of lactic acid bacteria in combination with melatonin and yeasts, such as saccharomycetes, for the preparation of dietary and pharmaceutical compositions to modify the composition of human intestinal flora, to stimulate the immune system and to improve intestinal and gynecological disorders. The present invention also relates to use of the composition of lactobacilli, melatonin and yeasts, in particular saccharomycetes, as a supplement to a food product.

### PRIOR ART

The microbiota is defined as a population of microorganisms that colonize a given environment and should not be confused with the term microbiome, which instead indicates the total genetic heritage possessed by the microbiota.

More specifically, “intestinal microbiota” means the overall ecosystem formed by fungi, viruses and bacteria that have adapted to live on the surface of the mucous membrane of the intestines or in the lumen thereof, developing immediately after birth, and influenced by the mode of delivery (vaginal vs. caesarean), initial nutrition (breastfeeding vs. artificial) and the host's genotype.

The fungi contributing to the mycobiota include species such as Candida, Saccharomyces, Aspergillus and Penicillium, and have an important role thanks to complex fungi-bacteria, fungi-fungi and fungi-host interactions influencing the host's health and in some cases diseases.

Viruses, which constitute the viroma, are present mainly in human feces, but there are also enteric viruses. Viruses are believed to affect the host's state of health by interfering with the structure of the bacterial community and its function, although it is not yet clear how this influence is exercised.

Bacteria are prokaryotic, that is unicellular organisms that have no nucleus; their activity in the human microbiota is mainly modulation of the immune system.

Three mechanisms have been identified by which the microbiota, through fermentation, can influence intestinal motility:
1. through the release of bacterial substances or the end products of bacterial fermentation;
2. through intestinal neuroendocrine factors;
3. indirectly, through the effects of mediators released by the intestinal immune response.

External factors (such as consumption of antibiotics, some substances introduced in diet, physical and psychological stress) and host-related factors can induce dysbiosis in the intestinal microbiota.

“Dysbiosis” therefore means disruption of the balance of the microbiota and, consequently, its normal functioning. This results in the selective suppression of some species in the microbiota leading to unregulated production of microorganism-derived products or metabolites that can become dangerous for the host, to the point of causing various disorders locally, systemically or even in more distant organs (Zhi Y. Kho and Sunil K. Lal—The Human Gut Microbiome—A Potential Controller of Wellness and Disease—Frontiers in Microbiology—August 2018, Volume 9, Article 1835).

The word “dysbiosis” was first adopted by the Russian scientist Elie Metchnikoff who hypothesized that the toxic compounds produced by the bacterial processing of food cause degenerative diseases (Michael T. Murray, Joseph Pizzorno—Treatise on Natural Medicine—Red Editions (2012)—pages 495-500).

The main causes of dysbiosis are:
Dietary disorders (a hyperprotein hyperlipid diet, rich in sugars and low in fiber; food allergies; malabsorption and impaired digestion of carbohydrates)
Poor digestive secretions
Stress
Antibiotic/pharmacological therapy
Weakened immune functions
Malabsorption
Intestinal infections
Altered pH

(Michael T. Murray, Joseph Pizzorno—Treatise on Natural Medicine—Red Editions (2012)-pages 495-500)

The symbiosis or pathogenicity of the 500 inhabitants of the human digestive tract is largely determined by the environment in which they live and the balance that is established between the various types of bacteria. Candida albicans is an example of an organism which, under normal conditions, lives in harmony with its host, but which, if it grows in an unbalanced way in relation to other intestinal microorganisms, can also cause serious problems.

One of the most interesting hypotheses regarding the imbalance and excessive growth of various organisms is the nature of the level of digestion of carbohydrates. The presence of undigested and unabsorbed carbohydrates within the large intestine and/or colon gives rise to the increased fermentation and excessive growth of some toxic species of bacteria. This leads to increased production of not only gas, but also short-chain organic acids, such as lactic acid, which are harmful to the intestinal mucosa. The damage caused to the latter exacerbates the problem of the decrease in the level of undigested disaccharides. Worsening of the damage to the gastrointestinal system generates increasingly abundant mucous secretions, which further remove carbohydrates from their digestive sites.

One of the possible treatments of dysbiosis is the “specific carbohydrate diet” which breaks the vicious circle of the non-digestion of some carbohydrates, permitting only the ingestion of pre-digestible or easily digestible carbohydrates wholly absorbed by the duodenum, making them inaccessible to distal bacteria. Basically, all disaccharides (wheat, rice, etc.) and all starchy vegetables (e.g. potatoes), as well as most of the starches present in legumes, are ruled out. Simple sugars such as glucose and fructose (present in honey, fruit and some vegetables) and dairy products containing hydrolyzed lactose are instead permitted (Michael T. Murray, Joseph Pizzorno—Treatise on Natural Medicine—Red Editions (2012)—pages 495-500).

However resort to diets is often not appreciated by patients, which is why there is a need to provide a drug or a dietary supplement that helps reduce discomfort and enables the negative effects of dysbiosis to improve.

As in the intestine, a “microbiota” is also present in the vaginal environment and “dysbiosis” can similarly occur. Such imbalances in the flora can compromise its physiological protective function and give rise to inflammatory phenomena. Vulvovaginal inflammations are inflammatory processes affecting the vagina with frequent involvement of the vulva and other areas of the lower female genital tract: cervix, urethra, bladder. Vulvovaginal inflammations are divided into bacterial, aerobic and fungal.

Inflammation may be caused by etiological agents from the endogenous bacterial reservoir (vaginal and rectal) or alteration of the vaginal microenvironment (bacterial vaginosis, aerobic vaginitis, fungal vaginitis).

The parts of the female genital apparatus in which pathogens preferably develop are the urethra, periurethral glands and cervix for Chlamydia and Gonococcus, the urethra, periurethral glands, uterine cervix and vagina for Mycoplasma hominis, the urethra, periurethral glands, uterine cervix and vagina for Ureaplasma urealytycum, mainly the vagina for Gardnerella, and the vulvovaginal zone for Candida.

Biofilms are structured communities of bacterial cells, often of different species, including fungal ones, enclosed in a self-produced polymer matrix and attached to an inert or living surface (A. Graziottin, Cystitis and recurrent vaginitis: role of biofilms and persister Cells. From pathophysiology to new therapeutic strategies, Minerva Ginec., 2014, vol.66, no. 5, p. 497-512).

In the vaginal environment, under physiological conditions and during the age of fertility, 90% of the microbial flora usually present in mutual symbiosis with the host are lactobacilli that can organize themselves into physiological biofilms and represent the healthy characterizing aspect of the vaginal ecosystem, while the remaining 10% are saprophytic bacteria.

Imbalance in this resident microbial flora is the source of recurrent uro-gynecological infections that constitute a serious medical problem, as at present there are no truly effective treatments (D. H. Martin, The microbiota of the vagina and its influence on women's health and disease. Am J Med Sci 2012; 343:2-9).

Altered bacterial flora can give rise to the generation of pathogenic biofilms, found in two main locations—extracellular and intracellular.

Extracellular polymicrobial biofilms, mainly located in the vagina, consist of an exopolysaccharide matrix (EPS) that acts as a protective system passively opposing the penetration of drugs and the effects of the immune response. These biofilms can also be organized on the surface of inert media, such as medical devices.

Biofilms, both supramucosal and inert, are coming to be of significant importance in gynecology and urology in many recurrent infections.

Intracellular biofilms, mainly located in the bladder, are characterized by a uropathogenic strain of Escherichia coli (Uro Pathogenic Escherichia coli, UPEC) carrying the K antigen and responsible for 75-85% of recurrent cystitis. UPEC invades the cells of the urothelium to form Intracellular Bacterial Communities (IBCs).

A rich polysaccharide matrix, surrounded by a protective uroplakin shell, envelops the intracellular bacterial communities, which are always ready to reactivate to damage urothelial cells, reinfect the urine and attack the urothelium at other points and cause chronic inflammatory phenomena in the bladder wall, resulting in “painful bladder syndrome”.

If not promptly diagnosed and adequately treated, the infection and the chronic inflammation that accompanies it can cause irreversible disruption of the cell architecture of the bladder wall up to vaginal dysbiosis and corresponding destruction of the microflora (as diagrammatically illustrated in FIG. 1).

Candida albicans is the most common fungal biofilm in gynecology, but it is also extremely difficult to treat. One study examined the efficacy of a group of antifungals (including prescription antifungals such as Nystatin and Diflucan) against Candida biofilms.

Researchers found that antifungals were initially effective against Candida, but with growth of the biofilm they became less and less effective. In fact, after 72 hours of biofilm growth C. albicans cells were highly resistant, leading to the conclusion that drug resistance develops over time, coinciding with maturation of the biofilm.

Other studies refer to an almost total resistance to antifungal agents by Candida associated with biofilm.

Candida can also migrate into the oral cavity and this area can also be altered by the presence of pathogenic biofilms.

Changes in the vaginal ecosystem are due to immuno-inflammatory factors (e.g. presence of inflammatory cytokines), hormonal factors (hormonal imbalances in the various stages of life, fertility, menopause, pregnancy), dysmetabolic conditions, antibiotic therapies and corticosteroid therapies.

Currently the most commonly used drugs for vaginal problems are azole drugs administered orally and topically. These include: Clotrimazole, Miconazole, Fluconazole, and their inappropriate use causes drug-resistance.

Azole resistance is a complex phenomenon mainly caused by multiple point mutations or the over-expression of ERG11, but also by genes (CDR1, CDR2) that code the outflow pumps for antifungal drugs.

The search for therapeutic alternatives for the treatment of Candida albicans infections is becoming increasingly difficult due to the higher resistance of biofilm. There is therefore felt a need to have available an effective treatment to induce low rates of resistance, reduce the frequency of recurrence, achieve high rates of eradication and obtain benefits for physiological biofilm.

It is well known that in the treatment of Candida-induced Vaginitis, both those uncomplicated and those complicated by the presence of biofilm, the use of Probiotics restores the healthy vaginal physiological biofilm/ecosystem, prevents resistance to antibiotics and can be associated with drugs to improve their action.

Saccharomyces cerevisiae (Meyen ex E. C. Hansen, 1883), an osmophilic unicellular organism belonging to the fungal realm, is a well-known species of yeast of the family Saccharomycetaceae. It has been scientifically proven that Saccharomyces CNCM I-3856 is capable of protecting the vaginal flora against pathogenic yeasts and bacteria.

Lactobacilli are facultative anaerobic or micro-aerophilic Gram-positive rod-shaped bacteria. In nature there are at least 60 species and they make up the majority of the group of lactic bacteria, so called because almost all their members convert lactose and other sugars into lactic acid through lactic fermentation. They are very common and usually non-pathogenic. In humans they are present in the vagina and gastrointestinal tract, where they are symbiotic and form a small part of the human microbiota.

Lactobacilli mainly produce lactic acid by fermenting sugars, reducing the pH of the environment in which they grow. Acidification of their environment inhibits the growth of some pathogenic microorganisms. This function is found in the vagina, where Lactobacillus constitutes 97%-98% of the normal microbial flora and prevents the proliferation of other microorganisms by maintaining the pH at values around 5.

Candidiasis occurs when Lactobacillus colonies die, due either to poor nutrition or antibiotic therapies, and the micete Candida albicans, constituting 2%-3% of the normal flora, proliferates excessively. There is therefore felt a need for a non-invasive, safe and effective approach, without side effects, to counter the effects of dysbiosis in general, and vaginal and intestinal infections, produced by mainly Candida albicans.

The vaginal ecosystem was for many years considered a static, homogenous environment consisting essentially of Lactobacillus (formerly known as Döderlein's bacillus), an anaerobic Gram-positive bacterium.

Any variations from this typical make-up were considered pathological. However, it subsequently became clear that this ecological niche contains a range of microorganisms in dynamic equilibrium, and that the host environment and its composition undergo frequent major changes over the course of a woman's life. It is affected not only by natural variations in the hormone profile, such as circulating levels of oestrogens, but by other important factors such as sexual activity, pregnancy, intimate hygiene, systemic diseases, pharmacological treatments (antibiotics, immunosuppressants), radiotherapy and traumas.

At birth, while descending the vaginal canal during labour, the neonatal vagina is colonized by pre-selected microorganisms from the maternal vaginal environment; in contrast, delivery via caesarean section leads to a broader, less predictable range of bacterial species acquired from other sources. The effect of maternal and placental oestrogens persists for some days after birth and the neonatal vaginal epithelium maintains the same histological structure as in its last weeks of foetal life, meaning it is thickened, trophic and high in glycogen.

This promotes the growth of lactobacilli, which is facilitated in turn by the acidic environment the lactobacilli themselves create through their ability to metabolize glucose to lactic acid. Circulating levels of steroid hormones then drop rapidly and the neonatal vaginal mucosa becomes thin and atrophic, with a reduced cell glucose content. This causes a rise in the vaginal pH, which in turn leads to a drastic drop in acidophilic microorganisms and hence in the oxide reduction potential. In this way, the acidophiles are replaced by a mixed, predominantly anaerobic microbial population (Staphylococcus, Streptococcus, Corynebacterium, E. Coli), which will comprise the vaginal flora throughout infancy until puberty. (This mixed population will be seen again post-menopause, when the decline in ovarian function leads to a collapse of circulating oestrogen levels: on average from over 120 pg/ml to around 18 pg/ml.)

On reaching sexual maturity, circulating oestrogen levels rise once more and the vaginal mucosa undergoes structural changes (epithelial thickening, cell pyknosis and glycogen synthesis). This leads to acquisition of the microflora typical of reproductive age, dominated (95%) by lactobacilli.

The most common species are L. iners, L crispatus, L. gasseri and L. jenesenii, followed by L. acidophilus, L. fermentum, L plantarum, L. brevis, L. casei, L. vaginalis, L. delbrueckii, L. salivarius, L. reuteri and L. rhamnosus.

These microorganisms help boost the local immune defences through the creation of a biofilm which prevents the adhesion of pathogens, competition for metabolites and the production of various chemicals, such as hydrogen peroxide, bacteriocin and lactic acid. Lactic acid lowers the endoluminal pH, which is the most important factor in limiting vaginal colonization by exogenous microorganisms, restricting the growth of potential commensal pathogens and inhibiting the activity of bacterial virulence factors such as sialidase and mucinase, enzymes which play a crucial part in evading the local immune response.

The presence of lactic acid is closely correlated with vaginal trophism, and hence indirectly with oestrogen levels. It is mainly derived from the anaerobic metabolism of glucose by lactobacilli, as demonstrated by the predominance (over 50%) in vaginal secretions of D-lactic acid, an isomeric form which human cells are unable to synthesize; lactobacilli can produce both D- and L-isomers.

Another important defence mechanism is the hydrogen peroxide produced by some lactobacillus strains, mainly L. crispatus and L. jenseii. This is toxic to a large number of catalase/peroxidase-negative bacteria, including G. vaginalis, E. coli and S. aureus. The combined action of hydrogen peroxide, uterine peroxidase (produced by the cervix and the endometrium) and chloride and iodide ions also limits bacterial growth through the activation of polymorphonucleates, which exert their bactericidal action in the intercellular spaces between epithelial cells.

Lactobacilli produce biosurfactants, which reduce the proliferation of pathogens by preventing them from adhering to the vaginal epithelium. The lactobacilli also co-aggregate with pathogenic bacteria, surrounding them with a local microenvironment that contains a high concentration of antimicrobial substances, thus facilitating the lactobacilli's bactericidal action.

An understanding of the physiology of the vaginal microflora is essential for the accurate, thorough interpretation of the pathological profile of the various infections of the female genital tract, thus permitting a specific, effective treatment. Recent years have seen a rise in the number of studies investigating the efficacy of probiotics in the treatment and/or prevention of infections of the female lower urogenital tract, especially vulvovaginal candidiasis and bacterial vaginosis (BV), the most common infections in women of reproductive age. The need for alternative treatment approaches arises from the frequent finding in clinical practice of recurrent infections. These may not only have a financial impact but can also cause both psychological and physical discomfort, thus affecting the patient's quality of life.

The failure of the antimicrobial treatments commonly used to treat these infectious diseases is related to two main factors. First, the improper use of over-the-counter medicines as self-medication, as increasingly advertised on blogs and forums targeted at women, has led to the development of antimicrobial resistance. Second, the negative impact of antimicrobials themselves on the vaginal microflora complicates the regeneration of lactobacilli—the vaginal ecosystem's first line of defence in women of reproductive age. Alteration of the microflora both facilitates colonization by exogenous microorganisms and enables potentially pathogenic commensal bacteria, normally present at low concentrations, to become virulent. To prevent and combat the onset of these infections, it is therefore important to restore the equilibrium of the local ecosystem. For this reason, the administration of exogenous lactobacillus preparations for both topical and oral use was proposed and evaluated.

The criteria used for the selection of strains for probiotic use in urogenital infections were first proposed by Reid in 1987 and were later modified by McLean. The main properties lactobacilli need to be suitable for use as probiotics are:
Presence of an amount sufficient to ensure an adequate degree of vaginal colonization; this is also influenced by the strain's ability to adhere to the epithelium. In relation to route of administration, both topical administration through vaginal capsules and oral administration are equally effective in determining adequate vaginal colonization. Oral formulations must be able to pass intact through the gastrointestinal tract to the rectum, from which the lactobacilli colonize the vaginal cavity;
Ability to adhere to the urogenital epithelium to form a biofilm coating the walls of the local mucosa. This both helps consolidate their colonization and protects the mucosa from infectious microorganisms by stopping them from forming their own film, which would increase their antimicrobial resistance;
Adequate antibacterial activity: specifically, they must be able to maintain a vaginal pH <4.5, which is essential for their subsequent replication and consequent production of antimicrobials such as bacteriocin and hydrogen peroxide. The probiotic activity of lactobacilli differs not only by species but is also strain-specific;
Survival at non-physiological, or in any case unideal, pHs and temperatures;
Resistance against the antimicrobials used to treat urogenital infections.

To date, scientific evidence on therapeutic efficacy in the clinical management of these infections is only available for some strains, namely L. rhamnosus GR-1, L. acidophilus, L. acidophilus LA02, L. fermentum B 54 orRC-14 (also known as L. reuteri RC-14), L. fermentum LF10 and L. plantarum P17630. Various clinical studies have shown that these strains are able to colonize the vagina and re-establish the equilibrium of the various microbial species normally present therein.

In relation to vulvovaginal candidiasis, evidence published to date reveals that some specific strains, namely L. rhamnosus GR-1 and L. fermentum RC-14 (L. reuteri RC-14), are effective against C. albicans and are a valid alternative to traditional prophylactic antifungal treatments. Numerous in vitro studies have shown that L. delbrueckii, L. plantarum, L. acidophilus and L. gasseri can inhibit the adhesion and/or growth of C. albicans through the production of biosurfactants or bacteriocin-like substances. In a 2006 review of eight studies (conducted between 1975 and 2006) investigating the use of probiotics in limiting vaginal colonization by yeast and in preventing the recurrence of such fungal infections, only two did not demonstrate any positive effect of lactobacilli in limiting the number of episodes. It should be borne in mind that these studies presented a number of limitations: not all included a control group, some had a low sample size and in most cases the diagnosis was based on the patient's own history, and was not confirmed by a culture test.

One reason for the great variability of the results reported by the numerous clinical studies conducted to date could be the high heterogeneity of the microorganisms investigated and the test protocols used. By analysing the available studies carefully, it can be seen that the benefits of probiotics are strictly strain-specific and correlated with the type of infection being treated, as well as patient variables (age, hormone status, genetic predisposition, sexual habits, intimate hygiene), the treatment duration and the type of product used (dose, formulation, route of administration).

It is evident that all these variables make it difficult to interpret the data as a whole. Many meta-analyses draw the conclusion that there is insufficient evidence to recommend the use of probiotics to prevent or treat genitourinary infections, even though some strains have proven effective against some infections through partial studies in humans, as confirmed in vitro and in animal studies.

A careful review is thus needed of the collected data that analyses the effects of each probiotic strain separately, so that future research can be conducted on the most promising microorganisms on the basis of results obtained in vitro and in better designed clinical studies.

One line of investigation is what role lactobacilli might have in the restoration of the lactobacillus vaginal flora after conventional antifungal treatments, and if such a therapy might help prevent dysbiosis or recurrent infections. However, little data is available on their effectiveness in formal randomized clinical trials.

If not specifically excluded in the following detailed description, what is described in this chapter is to be considered as an integral part of the detailed description.

### SUMMARY OF THE INVENTION

One of the objects of the present invention is to provide new pharmaceutical and dietary compositions that are useful for the treatment of dysbiosis in general, and diseases of the vaginal, intestinal and oral mucous membranes and vaginal and intestinal infections produced by pathogenic biofilms and especially by Candida albicans.

Another object of the present invention is to provide new dietary compositions that can be used to supplement conventional treatments of dysbiosis in general, and diseases of the vaginal, intestinal and oral mucous membranes, and vaginal and intestinal infections, produced by pathogenic biofilms and especially by Candida albicans.

Another object of the present invention is to provide a food supplement that is effective in modifying and rebalancing the microbiota in general and the composition of the oral and vaginal and intestinal flora.

The use of the compositions of the present invention (also referred below as Microbio+) are particularly effective in cases of mycotic vaginitis.

Another object of the present invention is to provide new compositions including lactic acid bacteria in combination with melatonin and yeasts, in particular saccharomycetes, which can be used as drugs and as supplements for common commercially available food products.

These and other objects which will become evident during the following detailed description have been accomplished thanks to the inventors' discovery that compositions containing lactic acid bacteria in combination with melatonin and yeasts, in particular saccharomycetes, can be used to modify intestinal and vaginal flora and/or stimulate the immune system of a patient who needs it in order to improve the patient's condition and/or treat patients with vaginal, oral and intestinal dysbiosis.

Thus, in a first embodiment, the present invention provides pharmaceutical and food supplement compositions which contain lactic acid bacteria in combination with melatonin and yeasts.

In one preferred embodiment of the present invention the composition includes lactic acid bacteria, in particular Lactobacillus acidophilus, in combination with melatonin and saccharomycetes, in particular Saccharomyces cerevisiae.

In one preferred embodiment of the present invention, the composition is in the form of a kit comprising:
Saccharomyces cerevisiae in pre-dosed packages and/or pre-packaged dosing units, preferably as pure product, encapsulated for example in cellulose capsules;
lactic acid bacteria, preferably Lactobacillus acidophilus, and melatonin, preferably as pure products, mixed together and encapsulated for example in cellulose capsules, pre-dosed packages and/or pre-packaged dosing units; vitamins such as vitamin B6 and excipients may be added if necessary.

The presentations are suitable for simultaneous sequential or delayed administration, possibly in combination with antifungal/antibiotic drugs, to increase their effectiveness, but especially to reduce the risk of recurrence.

Further objects of the invention and its scope will be evident from the detailed description below.

### BRIEF DESCRIPTION OF THE FIGURES

FIG. 1 diagrammatically illustrates the change from vaginal eubiosis to dysbiosis in accordance with the key below:

(1.) the endogenous factor can lead to a pro-inflammatory environment in the vagina=favorable for opportunistic pathogens

(2.) Candida albicans adheres to the vaginal epithelium and changes to the pathogenic form

(3.) Production of SAP=enzymes involved in inflammation

(4.) parallel growth of pathogenic bacteria

(5.) induction in the vaginal epithelium and damage to the mucosa=inflammation

FIG. 2: Group differences with VHI at T3. The VHI indicates the presence at T3-group 4 of an uninflamed, intact mucosa with a normal secretion, normal hydration and pH less than 4.6 (score 25). T3-group 1 (placebo) has a score of 11,7 VHI that indicates the presence of an inflamed mucosa, not intact, with an excessive secretion, poor hydration.

FIG. 3: Comparison symptom itch at T3. Patients in group 4-T3 (a product containing Lactobacillus acidophilus LMG S-29159, Saccharomyces cerevisiae CNCM I-3856 and Melatonin+excipients) were the only ones to achieve statistical significance in the improvement of all the outcomes evaluated: itching, burning and vaginal discharge as well as the reduction of VVC recurrences.

FIG. 4: Comparison symptom burning at T3.

FIG. 5: Comparison symptom vaginal discharges at T3.

FIG. 6: Study of the effect of Microbio+ capsules in woman with uncomplicated vulvovaginal candidiasis.

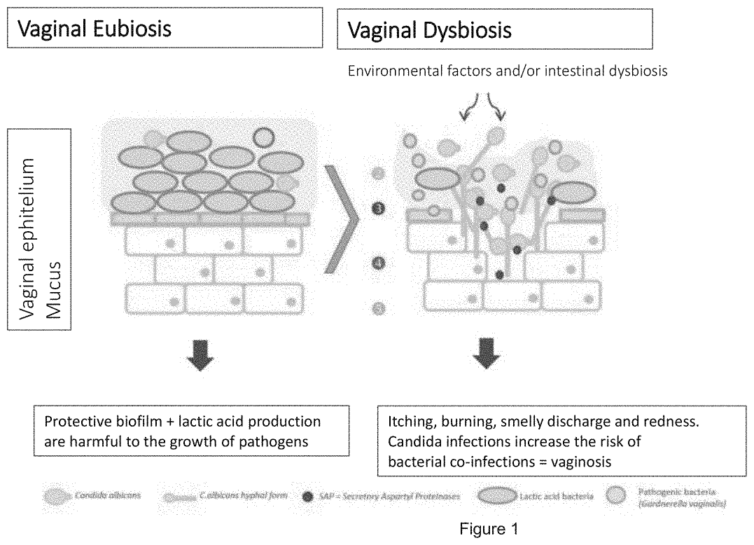

### DETAILED DESCRIPTION OF THE INVENTION

The term “lactic acid bacteria” refers to probiotic or ‘probiotically active’ organisms, that is organisms which can have a favorable effect on animal or human hosts. As used here, the term “lactic acid bacteria” refers to micro-aerophilic or anaerobic Gram-positive bacteria that ferment sugars with the production of acids, including lactic acid, as the predominantly produced acid, acetic acid, formic acid and propionic acid. The most industrially useful lactic bacteria are to be found among the Lactobacilli, Streptococci and Bifidobacteria.

The term “therapeutically effective quantity” as used here refers to the quantity of active ingredient intended to improve the well-being of an individual through ingestion of the active ingredient, in particular one that contributes to the ability of the composition to treat vaginal and intestinal dysbiosis.

The term “treatment” as used here refers to the partial or total elimination of vaginal and intestinal dysbiosis. The term “treatment” also includes reduction of the incidence of diseases related to vaginal and intestinal dysbiosis.

The terms “prevent” and “prevention” as used here refer to both preventing the onset of vaginal and intestinal dysbiosis and preventing the onset of a preclinically evident stage thereof. It also means prevention of the recurrence of dysbiosis.

The terms ‘dietary and/or dietary composition’ are to be considered to be equivalent to ‘nutraceutical’.

According to the invention, a pharmaceutical composition is a composition intended to restore the health of an individual and/or to prevent or cure a disease. In addition, a “pharmaceutical product” is to be considered as synonymous with “medication”.

According to the invention, an “active ingredient” is the causative agent of the beneficial, preventive or therapeutic effects of the composition according to the invention.

According to the present invention, the “active ingredient” as defined herein is the combination essentially comprising one or more lactic acid bacteria, preferably Lactobacillus acidophilus, one or more yeasts, in particular saccharomycetes, preferably Saccharomyces cerevisiae, and melatonin.

The pharmaceutical composition according to the invention may also include a pharmaceutically acceptable vehicle.

According to the invention, a “pharmaceutically acceptable vehicle” refers to any compound or group of compounds that are compatible for administration to an individual without significant adverse effects, intended to facilitate the administration or action of the active ingredients.

The term “individual” as used here refers to any human individual, primarily women, but also includes the elderly, children and infants affected by vaginal and intestinal dysbiosis.

Specifically, this invention relates to the prevention, treatment and alleviation of discomfort produced by vaginal and intestinal dysbiosis through the use of pharmaceutical and food compositions containing melatonin, a yeast, preferably belonging to the Saccharomyces family, and a bacterium from among those belonging to the Lactobacillus, Bifidobacterium and Streptococcus families. In particular the composition according to the invention can be used in the treatment of disorders associated with vaginal and intestinal diseases caused by the altered balance of flora (dysbiosis), and to reduce or eliminate inflammation due to local and systemic infections caused by the excessive growth of pathogenic microorganisms.

The invention relates in particular to food or pharmaceutical compositions containing as active ingredient the combination of melatonin with at least one strain of Saccharomyces cerevisiae and lactobacilli, in particular a strain of Lactobacillus acidophilus, for use in the treatment and prevention of vaginal infections, in particular those caused by the pathogen Candida albicans, reducing local inflammation, counteracting formation of the pathogenic biofilm and restoring the balance of the ecosystem, preventing any recurrent infections.

Lactic acid bacteria that can be used in the composition according to the invention are chosen from Bifidobacterium subsp. infantis, Bifidobacterium subsp. longum, Enterococcus faecium, Lactobacillus acidophilus, in particular LMG strain S-29159, Lactobacillus delbrueckii subsp. bulgaricus, Lactobacillus helveticus, Lactococcus lactis, Streptococcus thermophilus, Lactobacillus fermentum, Lactobacillus rhamnosus, Lactobacillus brevis, Lactobacillus casei, Lactobacillus plantarum, Lactobacillus kefir, Streptococcus lactis, and corresponding combinations. The strain Lactobacillus acidophilus LMG S-29159 is commercially available, and is for example marketed by DANISCO.

Yeasts that can be used in the composition according to the invention are chosen from those of the genus Saccharomyces, such as Saccharomyces lactis, Saccharomyces cerevisiae, Saccharomyces fragilis, in particular Saccharomyces cerevisiae CNCM I-3856, and corresponding combinations. Saccharomyces cerevisiae CNCM strain I-3856 is commercially available, and is for example marketed by LESAFFREHUMAN CARE.
1. The concentration of lactic acid bacteria used here is preferably equal to or greater than 0.5×10⁹ CFU, more preferably between 1×10⁹ and 1×10¹⁰, where CFU means colony forming units. The concentration used for yeasts is preferably equal to or greater than 1×10⁹ CFU, more preferably between 1×10⁹ and 1×10¹⁰, where CFU means colony forming units. The concentration of melatonin is 1 wt %, preferably between 0.2 and 5 wt %, preferably 0.5-1.5 wt %, preferably 3-5 wt %, preferably 0.2-2 wt %, preferably 0.2-1 wt %, preferably 2-5 wt %, preferably 0.5-1,5 wt %.

In a preferred embodiment of the present invention the composition of the active ingredient according to the invention comprises the combination of Lactobacillus acidophilus LMG S-29159, Saccharomyces cerevisiae CNCM I-3856, preferably in a total bacterial concentration of between 2 billion and 20 billion CFU per gram of bacteria, and melatonin in a quantity of 1 mg, preferably 0.5-1.5 mg. The active ingredient is part of a pharmaceutical and/or nutraceutical composition and this composition also includes a pharmaceutically acceptable vehicle, in addition to the active ingredient.

The bacteria in the composition according to the invention may be alive, dried, freeze-dried, attenuated or killed (e.g. heated at 80° C. for 5 minutes), or be present as bacterial lysates, or in any probiotically active form, according to different embodiments of the invention. In the first case—live freeze-dried lactic acid bacteria—the composition can be used to modify the ecology of the intestine and/or vagina and/or mouth and to stimulate the immune system. In the second case, killed lactic acid bacteria and/or bacterial lysates can be used to stimulate the immune system alone or in combination with other immunomodulating substances, selectively stimulating the growth of beneficial flora.

The dietary or pharmaceutical composition according to the invention may further include other components such as at least one mineral, such as zinc or selenium, and/or at least one vitamin, such as the B vitamins, more particularly vitamin B6, and/or carbohydrates selected to modulate the intestinal and vaginal (prebiotic) flora.

The dietary or pharmaceutical composition according to the invention may be administered alone or in combination with antibiotics and antifungals and/or any other medication used to counter intestinal and/or vaginal and/or oral diseases due to the growth and proliferation of pathogenic species. Medications that can be used include, for example, azole drugs such as Nystatin, Diflucan, Clotrimazole, Miconazole, Fluconazole.

The composition according to the invention may be prepared in the form of a capsule comprising the active ingredient in the form of viable and/or probiotically active organisms. The method by which the probiotic dosage form in capsules can be produced is by techniques known to those skilled in the art, for example by filling the capsules. An example of such a process is provided in the examples where the capsules are filled with a dry composition that contains the probiotically active organisms alone and/or with a stabilizing agent which includes other various excipients that are in themselves known.

The capsule, usually of cellulose, is filled either directly with yeasts and/or freeze-dried bacteria, or filled with yeasts and/or freeze-dried bacteria mixed with the rest of the excipients (minerals, vitamins and carbohydrates), which are first sieved and homogenized. Pressure is then applied to effect closure of the two parts of the capsule.

Depending on the invention, capsules containing 10⁵, 10⁶, 10⁹ or even 10¹¹ or more CFU per g of probiotics may be prepared.

The mineral is selected from the following: calcium, chlorine, phosphorus, magnesium, potassium, sodium, sulfur.

The preferred carbohydrate in this invention is selected from the group of water-soluble, non-gelling fibers including fructo-oligosaccharides, non-starch or beta-glucan polysaccharides, fructans, inulins, lactitol, lactosaccharose, lactulose, pyrodextrin or soybean oligosaccharides.

The preferred vitamins are selected from the group of B vitamins (B1, Thiamine; B2, Riboflavin; B3, Niacin or nicotinamide; B5, Pantothenic acid; B6, Pyridoxine, Pyridoxal, Pyridoxamine; B8, Biotin; B9, Folic acid; B12, Cobalamin), Vitamin D, Vitamin E, Vitamin, A; Vitamin K; Vitamin C.

In addition to capsules, pharmaceutical forms suitable as a vehicle for the composition according to the invention may be dispersible powders and granules, lozenges, tablets or pills, gels formulated for absorption through the buccal or genital mucosa. Other formulations may be liquid formulations for oral or vaginal administration, such as capsules or douches, or liquid or semi-liquid or semi-liquid formulations such as gels and creams.

Another aspect of the invention comprises a kit including the pharmaceutical/dietetic composition according to the invention in premeasured disposable or multipurpose packages containing the composition subdivided as follows:
in a first compartment, yeasts preferably in the form of a capsule or aliquots of gel or cream, in pre-dosed packages;
in a second compartment, lactic acid bacteria and melatonin, preferably in the form of a capsule or aliquots of gel or cream, in pre-dosed packages;
instructions for use;

to allow simultaneous, sequential or delayed administration of the active ingredient.

One preferred method of administration is to take two capsules per day, one capsule taken from the first compartment and one capsule taken from the second compartment, preferably for a period of 15 days.

Another preferred method of administration is to apply the cream or gel twice a day to the oral and vaginal mucous membranes, an aliquot of cream or gel taken from the first compartment and an aliquot of cream or gel taken from the second compartment, preferably for a period of 15 days.

The therapy thus designed is effective, well tolerated and has no side effects.

INGREDIENTS of first compartment capsules
Saccharomyces cerevisiae; Coating agent: hydroxypropyl-methylcellulose, preferably Saccharomyces cerevisiae CNCM I-3856, capable of inhibiting pathogens, anti-inflammatory action.

INGREDIENTS of second compartment capsules
Lactobacillus acidophilus; Coating agent: hydroxypropyl-methylcellulose, preferably Lactobacillus acidophilus LMG S-29159, capable of protecting vaginal flora against pathogenic yeasts and bacteria
Melatonin, capable of inhibiting the biofilm produced by Candida
Optional additional ingredients Microcrystalline cellulose;
Fruit-oligosaccharides; Anti-caking agents; Silicon dioxide, Magnesium salts of fatty acids; Vitamin B6 (pyridoxine hydrochloride); Coloring agents, such as titanium dioxide.

Recommended dose and method of use:
Take one capsule from the first compartment per day, to be swallowed with a liquid (such as a glass of water), or apply the gel or cream, preferably in the morning before breakfast.
Take one capsule from the second compartment per day, to be swallowed with a liquid (e.g. a glass of water), or apply the gel or cream in the evening before bedtime.

The diseases that can be treated with the compositions according to the invention are those related to diseases of the oral cavity, intestinal and vaginal dysbiosis, such as vaginitis and bacterial vaginosis and candidiasis, in particular vaginal infections: mycotic vulvovaginitis, bacterial vaginosis, produced for example by Gardnerella vaginalis, fungal vaginitis caused for example by Candida albicans.

The intestinal and vaginal dysbiosis that can be treated with the compositions according to the invention are those due to:

Dietary disorders due to hyperprotein, sugar-rich, hyperlipid diet, low in fiber; food allergies; malabsorption and impaired digestion of carbohydrates

Disorders due to poor digestive secretions

Stress

Disorders due to antibiotic/pharmacological therapy

Disorders due to weakened immune functions

Malabsorption and intestinal infections

Altered pH of mucosa

The vulvovaginal inflammations that can be treated with the compositions according to the invention are those due to inflammatory processes affecting the vagina with frequent involvement of the vulva and other areas of the lower female genital tract: cervix, urethra, bladder.

Vulvovaginal inflammations are divided into bacterial, aerobic, fungal and can be caused by etiological agents from the endogenous bacterial reservoir (vaginal and rectal), changes in the vaginal microenvironment (bacterial vaginosis, aerobic vaginitis, fungal vaginitis).

The parts of the female genital apparatus in which pathogens preferably develop and which can be treated with the compositions according to the invention are the urethra, periurethral glands and cervix for Chlamydia and Gonococcus, the urethra, periurethral glands, uterine cervix and vagina for Mycoplasma hominis, the urethra, periurethral glands, uterine cervix and vagina for Ureaplasma urealytycum, mainly the vagina for Gardnerella, the vulvovaginal zone for Candida, painful bladder syndrome caused by chronic inflammation of the bladder, changes in the vaginal ecosystem due to immune-inflammatory factors, hormonal factors, dysmetabolic conditions, antibiotic therapies, and corticosteroid therapies.

The table below shows a diagram of Candida diseases.

| | |
|---|---|
| Symptoms | Vulvar and vaginal itching, burning, dyspareunia, |
| | increase in vaginal secretions |
| | It is important to emphasize that these are non-specific |
| | symptoms, common to |
| | other infectious and non-infectious diseases (herpes |
| | simplex, trichomonas, |
| | bacterial vaginosis, chlamydia, gonococcus, contact |
| | dermatitis, psoriasis) |
| Signs | Vaginal and rectal erythema |
| | Frequently vulvar dermatitis |
| | Vaginal secretions with non-specific characteristics |
| | (whitish or yellowish color; |
| | formation of dense adhesive plaques) |
| Bacterioscopic | Vaginal fluid pH 4.5 |
| and laboratory | Fresh microscopy: leukocytes, spores and/or hyphae |
| findings | Positive culture for Candida |

The compositions according to the invention are also suitable for the prevention and treatment of oral diseases, especially those produced by Candida.

The compositions according to the invention can be administered in combination with other drugs such as antibiotics, anti-inflammatories, antiseptics, antifungals, in particular in combination with azole drugs such as Clotrimazole, Miconazole, Fluconazole, Nystatin and Diflucan.

The compositions according to the invention are also suitable for administration to human beings, including the elderly, children and the newborn, women being particularly preferred.

The compositions of the invention were found to be particularly effective in the treatment and improvement of conditions linked to the pathologies of the vulva and vagina.

Leucorrhoea and vulvovaginal itching and burning are typical symptoms of a genital infection involving Candida or aerobic and anaerobic bacteria. However, the appearance of symptoms, the ever greater resistance to azoles or antibiotics and the side effects commonly induced by some topical applications all point to the need for well-tolerated alternative treatments. To this end, efficacy and tolerability could be achieved by creating an oral preparation designed to combat uncomplicated forms of vulvovaginal candidiasis by acting as a sort of broad-spectrum supplement, as well by modulating the inflammatory response, that contained the following active ingredients:

Saccharomyces cerevisiae (CNCM I-3856) live strains

Melatonin

Lactobacillus acidophilus GLA-14 (LMG S-29159)

The different actions of the active substances were differentiated by designing a night & day formula involving the administration of two different daily formulas for 15 days, with the possibility of a maintenance cycle of at least 5 days in the premenstrual phase (typically the fungal reactivation phase):
1 tablet containing Saccharomyces cerevisiae (CNCM I-3856) in the morning
1 tablet containing melatonin and Lactobacillus acidophilus GLA-14 (LMG S-29159) in the evening.

This differentiation aimed to optimize the multiple functions of the various active ingredients in harmony with the circadian rhythm, which can be disturbed by worsening of the infection at night.

The action of these ingredients can be described as follows:
1) Saccharomyces cerevisiae(CNCM I-3856) 500 mg:
Adheres to the urogenital epithelium, forming a biofilm which both helps consolidate the colonization of indigenous lactobacilli strains and protects the vaginal mucosa from attack by Candida, the cause of such infections;
Intervenes in the coaggregation mechanisms of Candida albicans species, inhibiting their adhesion to the epithelia of the vaginal mucosa and consequent pathogenicity and speeding their elimination;
Inhibits the fungal germination process;
Activates the non-specific immune response and reduces the formation of inflammatory cytokines.

2) MELATONIN 1 mg:
Inhibits the pathogenic biofilm induced by Candida;
Reduces intestinal and vaginal inflammation (action mediated on Toll Like Receptor 4), thus reducing proinflammatory cytokines (IL4 and IL6);
Regularizes the sleep/wake circadian rhythm, thus also modulating local inflammatory response (itching, burning etc.), which is typically exacerbated at night.

3) G-LA 14 Lactobacillus acidophilus (LMG S-29159) 3 10⁹ UFC:
Inhibits the production of proinflammatory cytokines (especially interferon-gamma);
Increases the release of anti-inflammatory cytokines (especially IL12).

The inventors examined the action of the composition according to the invention on patients with dysbiosis according to the protocols of the Pilot Studies described below in which four groups taking the following formulations were formed:
GROUP 1: Excipients only.
GROUP 2: Saccharomyces cerevisiae and melatonin, plus excipients.
GROUP 3: Lactobacillus acidophilus, Saccharomyces cerevisiae and melatonin, plus excipients.

GROUP 4: Lactobacillus acidophilus LMG S-29159, Saccharomyces cerevisiae CNCM I-3856 and melatonin, plus excipients.

The inventors found a surprising improvement in the symptoms of patients treated with the composition containing Lactobacillus acidophilus LMG S-29159, Saccharomyces cerevisiae CNCM I-3856 and melatonin, plus excipients, than was found in patients given only the placebo composition. In particular, patients in this treatment group showed a significant decrease in symptoms associated with Candida albicans infections and in general discomfort due to dysbiosis-related disease.

The inventors also found a high and unexpected improvement in patients in groups 2 and 3. This unexpected improvement is evidence of a synergistic and not an additive interaction between the components of the composition, capable of producing much more obvious effects than would have been expected from taking the individual components not in combination with each other.

The composition according to the invention is therefore able to induce an unexpected improvement in dysbiosis conditions.

Candida vulvovaginitis is a symptomatic inflammation of the vulva and vagina, caused by fungi belonging to the Candida's genus: it is a common pathology responsible for most of the female genital infections that tends to recur.

Multiple causes responsible for vulvovaginal candidiasis: Candida is a saprophyte fungus that creates damage under certain conditions, turning to opportunistic (pathogenic).

The predisposing factors of the recurrent Candida vulvovaginitis are: antibiotics, intake of estrogen progestins, diabetes mellitus and pregnancy.

Candida vulvovaginitis generally begins with itching accompanied by burning, constant discomfort, pain during intercourse (dyspareunia) and irritation, and then, it degenerates into pain during urination, unbearable itching, creamy white leaks, sometimes smelly.

The vaginal mucosa is colonized by different bacterial species that contribute to create the physiological microflora.

In healthy women most of the flora is 90% lactobacilli and the remaining 10% is saprophytic bacteria. However, different factors can alter the balance of the vaginal microflora (eg diabetes, immunosuppressive therapy, wrong habits, etc.) and cause infections. The antimicrobial therapy usually used, acts against the microorganisms, both pathogenic and on the normal microflora, predisposing to new infections, with the risk of RECURRENCES.

During relapses it is recommended to use SELECTED Lactobacilli, which are able to restore the balance of the vaginal microflora and help neutralize/destroy so-called “pathogenic biofilms”.

In recent years there has been a growing increase in studies aimed at verifying and measuring the efficacy of probiotics in the treatment and/or prevention of infections of the lower female urogenital tract, particularly with regard to Candida vulvovaginitis and bacterial vaginosis (Bacterial Vaginosis, BV) which are the most common conditions affecting women of childbearing age. The need for alternative therapeutic approaches arises from the frequent recurrence in clinical practice of recurrences that entail an important psychophysical discomfort for the patient, significantly altering the quality of life with not negligible health costs. The failure of antibiotic therapy commonly used in the treatment of these infectious diseases is linked to two main factors such as: the development, by pathogenic microorganisms, of resistance to chemotherapeutics favored in recent years by the widespread use of over-the-counter drugs increasingly advertised on women's “blogs” and “forums” that improperly promote their use as self-medication, and the negative impact of antibiotics on vaginal microflora, thus complicating the regeneration of lactobacilli that constitute the main line of defense of the vaginal ecosystem in women in fertile age.

In order to prevent and counter the onset of these infections, it is therefore important to restore the balance of the local ecosystem: for this purpose the administration of exogenous lactobacilli in preparations for both topical and oral use has been proposed and evaluated.

The main characteristics that lactobacilli must have to be suitable for use as probiotics are:

a quantity that guarantees a sufficient degree of vaginal colonization which is also influenced by the ability of the strain to adhere to the epithelium. Regarding the mode of administration both the topical route, using vaginal capsules, and the oral route are equally effective in determining adequate vaginal colonization by these microorganisms. In particular the oral formulations must be able to maintain their integrity during the passage in the gastrointestinal tract up to the rectum, from where the lactobacilli, by ascension, access the vaginal cavity.

the ability to adhere to the urogenital epithelium forming a biofilm that covers the walls of the local mucosa; this favors on one hand the consolidation of their colonization and on the other it protects the mucosa from the aggression of the micro-organisms responsible for the infections, in particular preventing them from being in turn this “film” responsible for a higher antibiotic resistance.

adequate antibacterial activity; in particular they must be able to maintain a vaginal pH <4.5, an indispensable factor for their subsequent replication and the consequent production of antimicrobial substances such as bacteriocins and hydrogen peroxide. The probiotic activity of lactobacilli differs not only by species, but is a specific strain.

survival at a pH and at a temperature different from the physiological one or however not optimal for the lactobacilli.

the ability to resist antibiotics used in the treatment of urogenital infections.

Regarding Candida vulvovaginitis, from the data so far present in the literature, it emerges that some specific strains of Lactobacilli (such as rhamnosus GR-1, fermentum RC-14 also known as RC-14 reuteri) are effective against Candida albicans constituting, in particular, a valid alternative to classical anti-fungal prophylactic therapy. In fact, numerous in vitro studies have shown that Lactobacilli such as delbrueckii, plantarum, acidophilus, gasseri are able to inhibit the adhesion and/or growth of Candida albilcans through the production of biosurfactants or similar bacteriocin substances. Clinical studies in a 2006 review in which 8 studies (from 1975 to 2006) were selected on the usefulness of probiotics in limiting vaginal colonization by yeast and in preventing relapses of these fungal infections, only 2 of the studies considered to have shown no positive effect of lactobacilli in limiting the number of episodes. It should be borne in mind that these studies have different biases: in some the control group was not present, in others the sample examined was small and in most cases the diagnosis was based on the patient's amnesty data without being confirmed by a culture test.

One reason for the wide variability of the results reported by the numerous clinical studies carried out to date can be found in the fact that the microorganisms and the analysis protocols used are very heterogeneous. By carefully analyzing the available studies it can be observed that the benefits of probiotics are strictly strain-specific and related to the type of infectious pathology treated, as well as to the characteristics of the patient (age, hormonal conditions, genetic predisposition, sexual habits, health and hygiene conditions) to duration of treatment and the type of product used (dose and formulation, route of administration). It is clear that all these variables make the overall interpretation of the data difficult; many meta-analyzes draw the conclusion that there is not enough evidence to recommend the use of probiotics in the prevention or treatment of genitourinary infections, although some strains have proven to be effective in the context of some pathologies through partial human studies and confirmed in vitro and in the animal. Therefore, a thorough review of the collected data is required, which analyzes the effects of each probiotic strain separately, so that in the future research can be conducted on more promising microorganisms based on the results obtained in vitro and in better designed clinical trials

The compositions according to the invention are particularly effective in treating and improving conditions related to diseases of the vulva and vagina.

The following examples are illustrative and do not limit the scope of the invention.

### EXAMPLES

### Preparation of Capsules

The stages in the preparation of a composition according to the invention in capsule form are indicated in the flow diagram below.

### Specimen Composition

| WHITE CAPSULE | | Functions of the |
|---|---|---|
| INGREDIENTS | mg | ingredients |
| MICROCRYSTALLINE | 186.60 | Excipient |
| INSTALLATION FG102 | | |
| FRUCTO OLIGOSACCHARIDES | 50,00000 | Excipient |
| MAGNESIUM SALTS OF FATTY | 9.00 | Anti-caking agent |
| ACIDS | | |
| MELATONIN | 1.00 | Active |
| SILICON OXIDE | 5.00 | Anti-caking agent |
| LACTOBACILLUS ACIDOPHILUS | 45.0 | Active |
| LMGS29159 | | |
| VITAMINB6 HCI (ROCOAT) | 3.40 | Active |
| Capsule | 95 | |
| Hydroxy-propyl-methyl cellulose | | Coating agent |
| (E4641 | | |
| Anatase titanium dioxide | | Coloring |
| Total weight | 395.00 | |

| TRANSPARENT | | Functions of the |
|---|---|---|
| CAPSULE INGREDIENTS | mg | ingredients |
| SACCHAROMYCES | 500 | Active |
| CEREVISIAE CNCMI3856 | | |
| Capsule | 95 | |
| Hydroxy-propyl-methyl | | Coating agent |
| cellulose (E464) | | |
| Anatase titanium dioxide(E171) | | Coloring agent |
| Total weight | 595.00 | |

### Kit

Pack consisting of 2 blisters each containing 15 tablets for use in gynecology for bacterial vaginitis and candidiasis in particular. The compositions of the capsules are indicated in the previous example:

1 blister (TRANSPARENT CAPSULES) with the following composition

| Nutritional information | Per daily dose | RDA % |
|---|---|---|
| Saccharomyces cerevisiae | 500 mg | — |
| CNCM I-3856 | | |

1 blister (WHITE CAPSULES) with the following composition

| Lactobacillus acidophilus | 3000 million CFU | — |
|---|---|---|
| LMG S-29159 | | |
| Melatonin | 1 mg | — |
| Vitamin B6 | 0.7 mg | 50% |

The indication of the product is (according to ministerial directives):

Unilen Microbio+ is a food supplement based on probiotic lactic fermentations and yeasts which promote equilibrium of the intestinal flora. Vitamin B6 helps normal function of the immune system.

### First Pilot Study

Vaginal Dysbiosis_Vaginitis_Candida albicans.

Foreword

The human body is colonized by billions of microorganisms of different species, collectively defined as “microbiota” They are beneficial and protective microorganisms, with a perfect balance between the various colonies that make it up. However if this balance comes to be imbalanced disturbances may occur in the affected apparatus, defined by the term DYSBIOSIS.

The most famous microbiota is the intestinal one, but for women it is also important to have a vaginal microbiota in excellent condition.

The intestinal and vaginal microorganisms can be in a condition of dysbiosis, promoting the development of the most discomforting diseases that are grouped in gynecology as vaginitis and vaginosis.

When there is a variation in the proportion of microorganisms that are normally present in the vagina we speak of “vaginosis”. Infection, on the other hand, is defined as “vaginitis” and can be caused either by the arrival of foreign microorganisms or by the “predominance” of a normal inhabitant, among which the main saprophyte is Candida albicans.

Candida albicans is an opportunistic yeast that causes over 90% of all vulvovaginal infections with symptoms related to burning, vaginal discharge, painful urination, dyspareunia.

Because of intrinsic resistance to antifungal drugs such as azole derivatives, the search for anti-Candida albicans agents with new pharmacological targets is considered to be the main therapeutic strategy to prevent and treat this type of infection.

Objective

The pilot study used “a new product” suitable for the systemic treatment of dysbiosis, with particular attention to vaginal dysbiosis induced by Candida albicans.

Its components include active ingredients such as Saccharomyces cerevisiae (direct action on Candida; aggregation, multiplication, virulence, clearance), Lactobacillus acidophilus (anti-inflammatory and bacteriostatic—aerobic and anaerobic), Melatonin (immunomodulating, anti-inflammatory, biofilmolytic action, normalizing the circadian rhythm and rebalancing from the metabolic point of view).

The product is also indicated for patients with intestinal dysbiosis, as the opportunistic yeast in question, Candida albicans, is also naturally present in the intestinal microbiota.

Patients with vaginal dysbiosis induced by Candida albicans who showed symptoms related to vaginitis were followed up. Each patient was associated with a card on which, in addition to the patient's identification, were reported instrument examinations, previous treatments, symptoms before treatment with the product, the checks after 15 days, 1 month and 3 months from the start of treatment with the product and then the final assessment and any patient comments on the use of this new substance.

Method

The VHI (Vaginal Health Index) of each patient was established before and after treatment.

Each individual was given a treatment plan to ensure that it was as uniform as possible for all.

Patients were divided into 4 homogeneous groups using a randomized and controlled study protocol and received respectively:

GROUP 1: product containing only excipients (placebo)

GROUP 2: product containing Saccharomyces cerevisiae and melatonin, plus excipients

GROUP 3: product containing Lactobacillus acidophilus, Saccharomyces cerevisiae and Melatonin, plus excipients.

GROUP 4: product containing Lactobacillus acidophilus LMG S-29159, Saccharomyces cerevisiae CNCM I-3856 and Melatonin, plus excipients

Each patient, regardless of the group to which they belonged, received the product in the quantity necessary for 3 months treatment in special unlabeled containers.

Instrument examinations were performed for a more precise diagnostic definition and to exclude other pathologies. The patients underwent vaginal swabbing and VHI assessment before treatment.

To evaluate the effectiveness of the product, in addition to the state of the mucosa (moderately inflamed/very inflamed) detected through VHI, three symptoms variously present in Vaginitis were monitored:

itching, using a scale going from absence to unbearable discomfort;

vaginal discharge present or not present in mild, medium or abundant form;

burning, using a scale going from absence to unbearable discomfort.

Final Results

Considering the objective results obtained for VHI and the course of the various symptoms during the 3 months of treatment, a final assessment could be made in these terms:

“This study highlighted the synergistic effect of the ingredients Lactobacillus acidophilus LMG S-29159, Saccharomyces cerevisiae CNCM I-3856 and Melatonin, demonstrating a reduction in the clinical symptoms associated with Candida albicans-induced vaginitis that was significantly higher in GROUP 4 than for the individual active ingredients.

Sometimes repopulating both the intestine and the vagina with specific probiotics can help to avoid recurrence, reducing or even preventing economic, sociological and psychological damage to patients.

The Vaginal Health Index (VHI) is a tool that doctors can use, evaluating 5 parameters (vaginal elasticity, vaginal secretions, ph, epithelial mucosa, vaginal hydration), to give individual scores to arrive at a final score that defines the state of vaginal health.

TABLE 1
Vaginal health index (Bachmann et al. [22]).

| | | Fluid secretion | | | |
|---|---|---|---|---|---|
| | Overall | type and | | Epithelial | |
| Score | elasticity* | consistency | pH | mucosa | Moisture |
| 1 | None | None | 6.1 | Petechiae | None, |
| | | | | noted before | mucosa |
| | | | | contact | inflamed |
| 2 | Poor | Scant, thin | 5.6-6.0 | Bleeds with | None, |
| | | yellow | | light contact | mucosa |
| | | | | | not |
| | | | | | inflamed |
| 3 | Fair | Superficial, thin | 5.1-5.5 | Bleeds with | Minimal |
| | | white | | scraping | |
| 4 | Good | Moderate, thin | 4.7-5.0 | Not friable, thin | Moderate |
| | | white | | mucosa | |
| 5 | Excellent | Normal (white | ≤4.6 | Not friable, | Normal |
| | | flocculent) | | normal mucosa | |

*Lower score corresponds to greater urogenital atrophy.

The Visual Analog Scale (VAS) is a response scale which allows to measure for subjective characteristics that cannot be directly measured. Generally speaking, when responding to a VAS item, respondents specify their level of agreement to a statement by indicating a position along a continuous line between two end-points. The scale ranges from 0 to 10 and, for example in case of pain, one end indicates the absence of pain, while the other end represents the worst pain imaginable. The scale is filled in by the patient, who is asked to draw a line on the line that represents the level of pain experienced. Less then one indicates absence of pain; from one to four the intensity of pain is defined as mild; four to six mild or moderate; from six to ten moderate or severe. Scores can be used to assess the effectiveness of a medical treatment, as in this case. In clinical work, the VAS scale is chosen because, despite the narrowness of the scale (or maybe because of it), most people quickly grasp what they are being asked and generally provide sensible approximations of their pain experience or any other simptomatology.

### Second Pilot Study

The present preliminary study (randomized and controlled) has as its main purpose to investigate the safety and efficacy of a new product for oral use to treat VVC vulvovaginal candidiasis and at the same time treat its symptoms.

The product under examination is an oral formulation prepared using active ingredients based on

live strains of Saccharomyces cerevisiae CNCM I-3856

melatonin

Lactobacillus acidophilus LMG S-29159

that would be able to correctly counteract uncomplicated forms of mycotic vaginitis, acting as a sort of broad-spectrum integrator also determining a modulation of the inflammatory response.

The action of these components could thus be defined:

1) Saccharomyces cerevisiae CNCM I-3856 e: \grave{ }

able to adhere to the urogenital epithelium forming a biofilm, on the one hand it favors the consolidation of the colonization of indigenous lactobacilli strains and on the other it protects the vaginal mucosa from the aggression of the genus Candida microorganisms responsible for these infections

able to act on the mechanisms of coaggregation of Candida albicans species by inhibiting their adhesion processes on vaginal mucosa epithelia and consequent pathogenicity, accelerating the clearance of the fungus

capable of inhibiting the fungus germination processes

able to activate the non-specific immune response and reduce the formation of inflammatory cytokines

2) MELATONINA is:

capable of inhibiting the pathogenic biofilm induced by Candida

reduce the inflammatory insult in the intestinal and vaginal mucosa (action mediated on Toll Like Receptor 4 with consegeunte reduction of proinflammatory cytokines (IL4 and IL6)

regularize the sleep/wake circadian rhythm and modulate in this sense the local inflammatory response which typically is exacerbated at night (prurotourism, burning etc)

3) Lactobacillus acidophilus LMG S-29159 \grave{ }:

Inhibit the production of proinflammatory cytokines (especially in relation to IFgamma)

Increase the release of anti-inflammatory cytokines (especially for IL12)

They were considered as main outcomes:

state of the mucosa (inflamed or not inflamed)

improvement of symptoms (itching, vaginal discharge and burning) reported by patients (using a scale from 0 to 10 for each symptom).

Patients not pregnant, without a history of recurrent genital infections (bacterial or fungal) and without chronic or dysmetabolic pathologies, suffering from acute vulvovaginitis caused by Candida albicans confirmed by microscopic/cultural examination and vulvovaginal symptoms (itching), were considered eligible for the study. leucorrhoea, burning).

After clinical evaluation (anamnesis and gynecological examination) the patients with the inclusion requirements were selected and they were randomized into 4 different groups:

GROUP 1: product containing exclusively placebo excipients.

GROUP 2: product containing Saccharomyces cerevisiae and Melatonin+excipients

GROUP 3: product containing Lactobacillus acidophilus, Saccharomyces cerevisiae and Melatonin+excipients

GROUP 4: product containing Lactobacillus acidophilus LMG S-29159, Saccharomyces cerevisiae CNCM I-3856 and Melatonin+excipients

Patients were evaluated at time T0 (time of enrollment), at time T1 (after 15 days of therapy), and at time T2 (after 30 days of therapy), and at time T3 (after 90 days of therapy) from a observer unaware of the assigned treatment

For each patient, the symptoms complained of itching, burning, vaginal discharge using a VAS scale (Visual Analogic Scale) were recorded at each visit: each patient indicated the severity of symptoms on a visual analogue scale, 10 cm long without interruptions from 0 to 10.

The selected patients were also subjected to a VHI in order to evaluate the state of the mucosa at time T0, T1, T2 and T3.

Statistic Analysis

The recorded data has been entered on a database built with Office Excel® software and analyzed with Office Excel software

Results

For each group 10 patients were recruited. The comparison of the average values (VHI data) recorded for each symptom and the average values of the mucosal status (VHI data) did not show significant differences between the various groups which are therefore homogeneous results. No adverse events or side effects were recorded in any group of patients and there were no drop-outs.

For each group the data at the various times T0, T1, T2 and T3 were compared.

For patients in group 1 (placebo) there were no significant differences in the comparison between the VHI values of the various symptoms or the state of the mucosa at various times; patients in group 2 (a product containing Saccharomyces cerevisiae and Melatonin+excipients) showed a statistically significant improvement in pruritus and losses, but not a burning sensation) in patients of group 3 (a product containing Lactobacillus acidophilus, Saccharomyces cerevisiae and Melatonin+excipients), instead, itching was significantly reduced, with no statistically significant improvement in the other symptoms.

Patients in group 4 (a product containing Lactobacillus acidophilus LMG S-29159, Saccharomyces cerevisiae CNCM I-3856 and Melatonin+excipients) were the only ones to achieve statistical significance in the improvement of all the outcomes evaluated: itching, burning and vaginal discharge as well as the reduction of VVC recurrences.

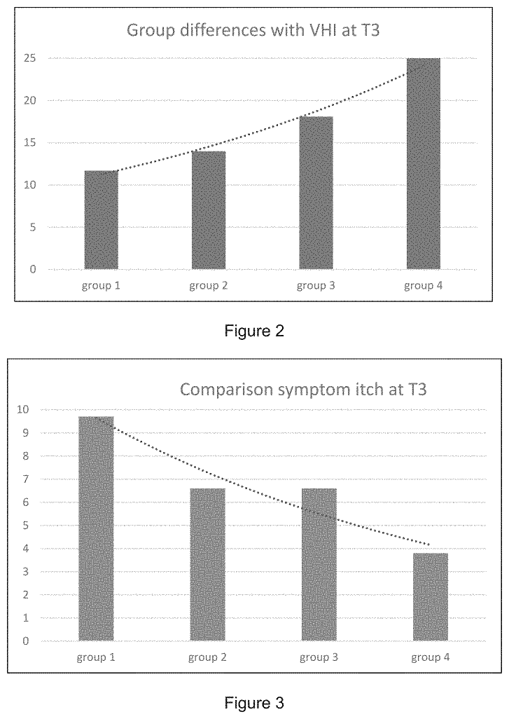

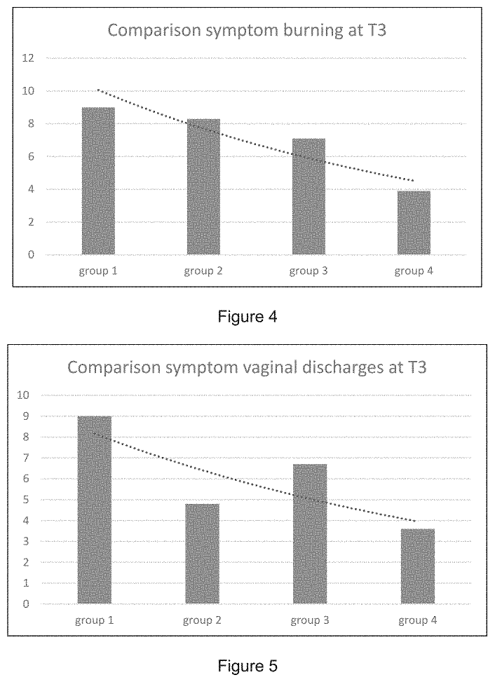

The results are shown in FIGS. 2-5.

Conclusions

This preliminary study has shown that the new oral product based on Saccharomyces cerevisiae CNCM I-3856 (effective in the control of vaginal Candida proliferation) Melatonin (capable of inhibiting Candida-induced pathogenic biofilm); Lactobacillus acidophilus LMG S-29159 (with anti-inflammatory activity) is safe and effective. Furthermore, as shown in the results, the synergistic effect of the active ingredients has shown a higher effect compared to the single active ingredients, or active ones but of different strains, allowing the group of treated patients to achieve a statistically significant improvement in all the outcomes analyzed.

A further study was conducted using the preferred composition of the invention with reference to a drug normally used in therapy.

### Third Pilot Study

Study of the Effect of Microbio+ CAPSULES in Woman with Uncomplicated Vulvovaginal Candidiasis.

Objective

The primary endpoints are:

reduction in frequency of recurrences

re-equilibrium of the vaginal microflora (through assessment of clinical signs and Lactobacillus grade) in women with uncomplicated vulvovaginal candidiasis.

Study design

Open-label

Investigational Treatments

CLOTRIMAZOLE group: single administration of topical antifungal treatment with no maintenance cycle

Group B: Microbio+ for 15 consecutive days and maintenance cycle of 5 days a month in premenstrual phase for 3 months

Inclusion and Exclusion Criteria

Inclusion Criteria:

Uncomplicated Vulvovaginal candidiasis;

Microscopic diagnosis of vulvovaginal candidiasis through wet mount or vaginal culture;

Age >18 years-55 years;

Not menopausal (defined as 12 or more months since last menstrual cycle at appropriate age);

Exclusion Criteria:

Complicated vulvovaginal candidiasis (recurrent and/or occurring in immunosuppressed subject, or chronic candidiasis) (NB: for the purposes of this study protocol, recurrent infections is defined as at least 4 culturally confirmed episodes in 12 months), infections in immunosuppressed subjects and chronic fungal infections are defined as complicated vulvovaginal candidiasis);

Systemic antibiotic or antifungal treatment, whether ongoing or in the 4 weeks prior to entering the study;

Pregnant or breastfeeding women;

Autoimmune diseases, thyroid diseases or history of atopy;

Diabetes mellitus;

Hypersensitivity to lactobacilli.

Methodology

The total study duration is 12 weeks.

Diagnosis (V0) and start of treatment

Day 15, day 30 and day 90 visits (V1, 2 and 3): At each visit, the patient was asked if she had taken the treatment regularly and if she had experienced any clinical symptoms or adverse events. A clinical examination, microscopic wet mount examination or (if available) cultural examination were performed to establish the effect of the treatment and the lactobacillus grade.

Sample Size

20 patients per arm

Rationale for the use of a supplement, Microbio+ containing saccharomyces cerevisiae(CNCM I-3856), melatonin and Lactobacillus acidophilus GLA-14 (LMG S-29159)

### Study Endpoint

Primary outcomes:
Evaluation of the efficacy of Microbio+ in women with acute VVC:
   1. Clinical cure rate (resolution of symptoms) of VVC at 15, 30 and 90 days;
   2. Microbiological and microscopic cure of VVC (negative vaginal culture or absence of hyphae at wet mount);
   3. Recurrence rate (defined as the presence of symptoms or positive fungal culture or presence of hyphae at wet mount) in 3-month follow-up period.

Secondary outcomes:
Safety and tolerability of Microbio+ in adult women.

Final Results

In the group treated with Microbio+, clinical and microbiological cure at 15 and 30 days was observed in 18 women (90%), compared with 16 women (80%) in the group treated with topical clotrimazole. The efficacy of Microbio+ in these uncomplicated forms was therefore not inferior to a single vaginal administration of the azole.

Only 4 women (20%) in the Microbio+ group presented symptomatic recurrences within the 3 months of follow-up, compared with 8 women (40%) in the clotrimazole group.

Microscopic wet mount analysis at 1 and 3 months demonstrated a significant increase in lactobacillus count and a reduction in the polymorphonucleate cells in the Microbio+ group.

This result supports the concept that the use of Microbio+ determines not only cure of uncomplicated fungal infections equivalent to the topical azole, but also the early (1 month) and long-lasting (3 months) re-equilibration of the vaginal microflora, reducing the fungal infection recurrence rate. This effect could be ascribed both to the ability of Microbio+ to modulate the inflammatory response (reduction in polymorphonucleates) and to the significant increase in the vaginal lactobacillus flora (booster effect).

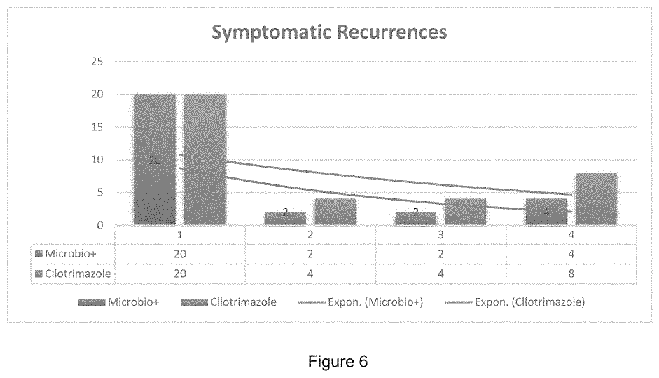

In brief, the results, illustrated in FIG. 6, can be summarized as follows:

Clinical and microbiological cure in 1 month (90% in Microbio+ Group vs. 80% in Clotrimazole group)

Statistically significant reduction in symptom recurrence rate in women using Microbio+ (20% vs. 40%)

Significant increase in lactobacillus bacteria and reduction in polymorphonucleates cells showed by wet mount at 1 and 3 months in women using Microbio+.

1. A composition comprising melatonin in combination with Saccharomyces cerevisiae CNCM I-3856 and Lactobacillus acidophilus LMG S-29159,
wherein the melatonin is in amount of between about 0.2 to 5 wt %.

2. The composition according to claim 1, wherein each of the Saccharomyces cerevisiae and Lactobacillus acidophilus strain are present in an amount equal to or higher than about 0.5 10⁹ colony forming units (CFU, or UFC).

3. The composition of claim 1, further comprising Saccharomyces lactis, Saccharomyces cerevisiae, Saccharomyces fragilis and corresponding mixtures.

3. The composition of claim 1, further comprising a lactic acid bacteria selected from any of Bifidobacterium subsp. infantis, Bifidobacterium subsp. longum Enterococcus faecium, Lactobacillus acidophilus, Lactobacillus delbrueckii subsp. bulgaricus, Lactobacillus helveticus, Lactococcus lactis, Streptococcus thermophilus, Lactobacillus fermentum, Lactobacillus rhamnosus, Lactobacillus brevis, Lactobacillus casei, Lactobacillus plantarum, Lactobacillus kefir, Streptococcus lactis and corresponding mixtures.

4. The composition of claim 1, further comprising: minerals selected from calcium, chlorine, phosphorus, magnesium, potassium, sodium, sulfur zinc, selenium; carbohydrates selected from water-soluble, non-gelling fibers including fructo-oligosaccharide, non-starch polysaccharides or beta-glucans, fructans, inulins, lactitol, lactosaccharose, lactulose, pyrodextrins, soy oligosaccharides; vitamins selected from the group of B vitamins (B1, Thiamine; B2, Riboflavin; B3, Niacin or nicotinamide; B5, Pantothenic acid; B6, Pyridoxine, pyridoxal, pyridoxamine; B8, Biotin; B9, Folic acid; B12, Cobalamin), Vitamin D, Vitamin E, Vitamin, A; Vitamin K; C vitamin; and mixtures of said components.

6. The composition of claim 1, wherein the lactic acid bacteria and the saccharomycetes are alive, killed and/or bacterial lysates.

7. The composition of claim 1, formulated as a food supplement.

8. The composition of claim 1, formulated as a pharmaceutical composition for use in the medical field.

9. A method for treating or preventing intestinal, oral and vaginal dysbiosis, or for relief of discomfort caused by intestinal, oral and vaginal dysbiosis, comprising administering to an individual in need thereof a composition of claim 1.

10. The method of claim 9 wherein the intestinal and vaginal dysbiosis is due to:
Dietary disorders (high-protein high-sugar hyperlipid low-fiber diet; food allergies; malabsorption and impaired carbohydrate digestion)
Poor digestive secretions
Stress
Antibiotic/pharmacological therapy
Weakened immune functions
Malabsorption
Intestinal infections, or
altered pH.

11. A method for treating or preventing any one of the conditions selected from: oral cavity conditions; vaainitis and bacterial vaginosis; candidiasis; vaginal infections such as mycotic vulvovaginitis; bacterial and fungal vaginosis; chronic inflammation of the bladder; vulvovaginal inflammation of the vagina with involvement of vulva and other areas of the lower female genital tract, such as the cervix, urethra, bladder, rectum, periurethral glands and vagina, comprising administering to an individual in need thereof a composition of claim 1.

12. The method of claim 9, wherein the composition is administered in combination with an azole drug,
wherein optionally the azole drug is selected from any one of: Nystatin, Diflucan, Clotrimazole, Miconazole, and Fluconazole.

13. The pharmaceutical composition of claim 8, manufactured or formulated in the form of a solid formulation or a liquid formulation for oral or vaginal administration,
wherein optionally the solid formulation is a capsule, a dispersible powder, a granule, a tablet, a pill, a gel,
and optionally the pharmaceutical composition is formulated for absorption through the buccal or genital mucosa;
wherein optionally the liquid formulation for oral or vaginal administration comprises an ova or a lavender formulation, or
and optionally the liquid formulation is formulated as a liquid or semi-liquid or semi-liquid formulation,
and optionally the pharmaceutical composition is formulated as a gel or a cream.

14. A dietary supplement suitable for administration to human beings, men or women, including the elderly, children and infants comprising a composition of claim 1.

15. A Kit comprising the following components in single or multi-use pre-dosed packages, comprising components divided as follows:
a first compartment comprising Saccharomyces cerevisiae CNCM I-3856, preferably in the form of a capsule or cream or gel, in pre-dosed packs;
a second compartment comprising Lactobacillus acidophilus LMG S-29159 and melatonin, preferably in the form of a capsule or cream or gel, in pre-dosed packs; and
Instructions for use;
optionally dosaged or compartmentalized for simultaneous, sequential or delayed administration.

16. The Kit according to claim 15, wherein administration is indicated in the doses comprising:
Take one capsule a day from the first compartment, or apply the gel or cream, in the morning; and
Take one capsule a day from the second compartment, or apply the gel or cream, in the evening.

17. The composition of claim 1, wherein the melatonin is present in the amount of between about 0.5 to 1.5 wt %, or between about 3 to 5 wt %, or between about 0.2 to 2 wt %, or between about 0.2 to 1 wt %, or between about 2 to 5 wt %.
